(19) United States
(12) Patent Application Publication
(10) Pub. No.: US 2019/0302052 A1
(43) Pub. Date: Oct. 3, 2019

(54) HANDHELD HEAVY METAL RAPID DETECTION SYSTEM

(71) Applicant: AccuSensing Technology Co., LTD., Taichung City (TW)

(72) Inventor: Han-Yu CHANG, Taichung City (TW)

(21) Appl. No.: 16/366,574

(22) Filed: Mar. 27, 2019

(30) Foreign Application Priority Data
Mar. 27, 2018 (TW) 107110563

(51) Int. Cl. G01N 27/416

(52) U.S. Cl. CPC G01N 27/4161; G01N 33/18

(57) ABSTRACT

A handheld heavy metal rapid detection system includes a detection strip, a detection key and a detection host. The detection system can be used to detect a variety of heavy metals in aqueous solutions, and is convenient for operators to carry out rapid detection by hand without the need to pre-treating water samples. The problems that water samples must be sent to professional laboratories for testing, which is time-consuming and expensive, can be mitigated. The detection strip and its matching detection key have the same color appearance for a more convenient identification. Misuse and detection errors can be prevented, and the detection accuracy can be improved.

### FIELD OF THE INVENTION

The present invention relates to a heavy metal detection system, and more particularly to a heavy metal detection system applicable for a variety of heavy metals.

### BACKGROUND OF THE INVENTION

Heavy metals can enter the human body trough diet but cannot be catabolized by the liver. They can be accumulated in the brain, kidneys and other organs and damage the normal function of the body. Common heavy metals include zinc, lead, nickel, copper, mercury, cadmium and chromium. While arsenic is a non-metal, many of its properties are similar to those of heavy metals, and are therefore generally classified as one of the heavy metals.

In order to prevent heavy metal pollution, it is necessary to establish a heavy metal detection technology in the environment. In the past, heavy metal detection relied on inductively coupled plasma atomic emission spectrometers, inductively coupled plasma atomic mass spectrometers, and atomic absorption spectrometers. Although these heavy metal detection technologies are mature, the inspection cannot be exploited at the sampling site. Besides, these equipments are expensive. The water sample needs to be pre-treated and requires professional technicians to operate. As a result, the detection time is long, the cost is high, and it is difficult to meet the needs of on-site inspection.

The present invention has arisen to mitigate and/or obviate the afore-described disadvantages.

### SUMMARY OF THE INVENTION

One of the aspects of the present invention is to provide a device capable of quickly performing on-site heavy metal detection.

To achieve the above and other aspects, the present invention provides a handheld heavy metal rapid detection system, which includes a detection strip, a detection key and a detection host. The detection strip has a heavy metal detection zone, a strip identification zone and a strip connection port. The heavy metal detection zone is adapted to be contacted with a solution to be detected and is adapted for electrochemical reaction with a target heavy metal. The strip connection port is electrically connected to the heavy metal detection zone. The detection key has a memory chip, a key identification zone and a key connection port. The memory chip is stored with detection parameters of the target heavy metal. The key identification zone has the same color as the strip identification zone. The key connection port is electrically connected to the memory chip. The detection host has a processor, a host screen, a first connection port and a second connection port. The host screen, the first connection port and the second connection port is electrically connected to the processor. The first connection port is adapted to be electrically connected to the strip connection port. The second connection port is adapted to be electrically connected to the key connection port. The processor is adapted to read the detection parameters from the memory chip, to activate the heavy metal detection zone based on the detection parameters, to acquire a detection signal from the heavy metal detection zone, and to show a detection result on the host screen based on the detection signal.

With the above technology, the operator can quickly detect a plurality of heavy metal concentrations at the sampling site by using the detection system of the present invention. The detection strip and the detection key of every individual heavy metal have the same color in appearance, which is convenient for the operator to identify and avoid misuse and detection errors. As a result, the accuracy of detection can be improved.

### BRIEF DESCRIPTION OF THE DRAWINGS

FIG. 1 is an explosive view of an embodiment of the present invention;

FIG. 2 is a front view of the embodiment of the present invention;

FIG. 3 is an exemplary detection procedure using the detection system of the embodiment of the present invention.

### DETAILED DESCRIPTION OF THE PREFERRED EMBODIMENTS

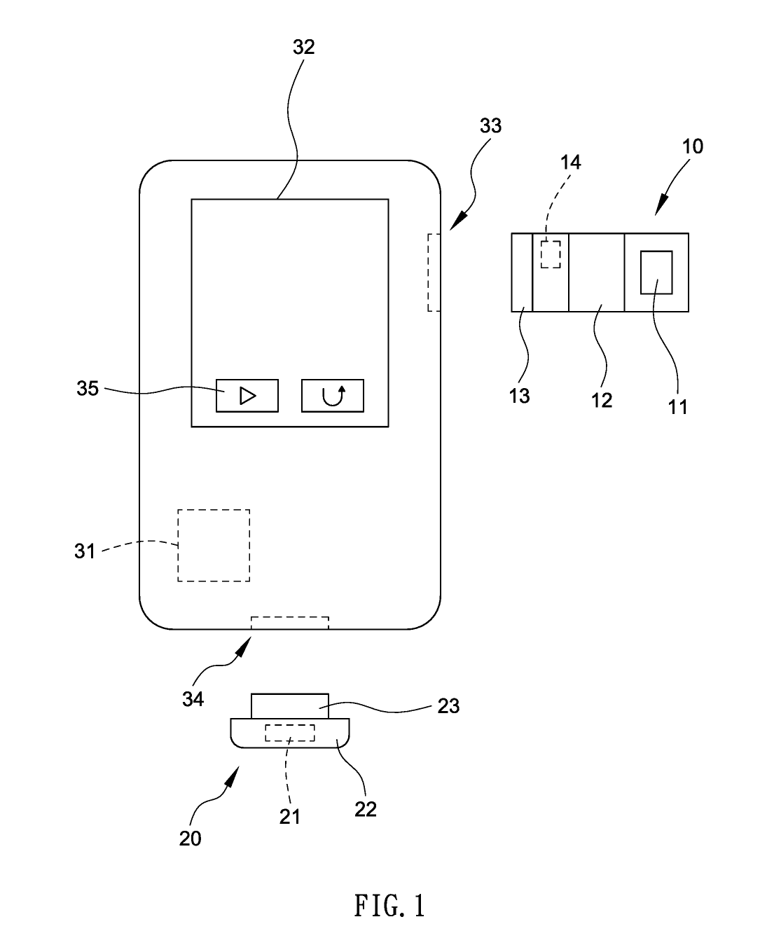

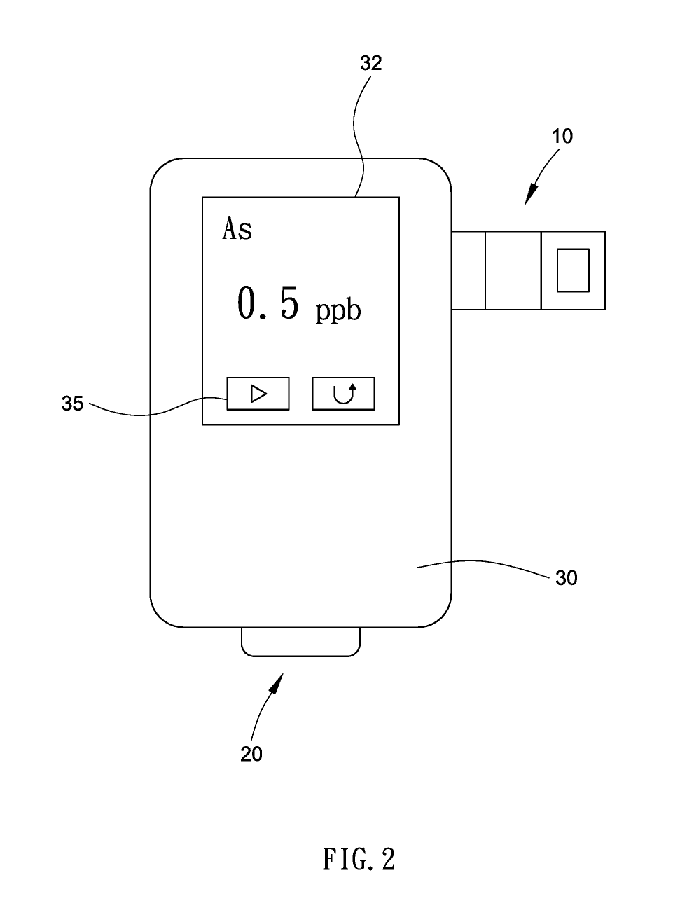

Please refer to FIGS. 1 and 2 for an embodiment of a handheld heavy metal rapid detection system (hereinafter referred to as detection system) of the present invention. The detection system includes a detection strip 10, a detection key 20 and a detection host 30. The detection system of the present invention can be applied to detections of a variety of heavy metals, including but not limited to arsenic, zinc, lead, nickel, copper, mercury, cadmium and chromium, by replacing different detection strips 10 and the detection keys 20 matched with the detection strips 10, respectively.

The detection strip 10 has a heavy metal detection zone 11, a strip identification zone 12 and a strip connection port 13. The heavy metal detection zone 11, for example, includes a working electrode, an auxiliary electrode and a reference electrode. The three electrodes are electrically connected to the strip connection port 13. The working electrode may be covered with reagent reactive with the target heavy metal or which facilitates the electrochemical reaction between the working electrode and the target heavy metal, so that the detection strip 10 does not need to be pre-treated before the detection and is convenient to use. The electrodes are generally encapsulated by a casing, in which the casing has an opening corresponding to the heavy metal detection zone 11 where the electrodes are exposed. The strip identification zone 12 is formed on the casing, for example. The heavy metal detection zone 11 can be immersed in the solution to be tested so that the possibly existing target heavy metal can electrochemically react with the electrodes of the heavy metal detection zone 11.

The detection key 20 has a memory chip 21, a key identification zone 22 and a key connection port 23. The memory chip 21 is stored with detection parameters of the target heavy metal and the identification information thereof according to the specific target heavy metal and the difference in manufacturing parameters of each batch of the detection strips. The memory chip 21 is encapsulated by another casing, on which the key identification zone 22 is formed, for example. The memory chip 21 is electrically connected to the key connection port 23. In the present embodiment, every target heavy metal is represented by a distinct identifying color. Different target heavy metals have different identifying colors. For two matching detection strip 10 and detection key 20, the strip identification zone 12 and the key identification zone 22 thereof have the same color. This helps the operator to identify whether the detection strip 10 and the detection key 20 are used to detect the same target heavy metal before the detection is performed, thereby avoiding the detection error and reducing the waste of the detection strip 10.

The detection host 30 has a processor 31, a host screen 32, a first connection port 33 and a second connection port 34. The host screen 32, the first connection port 33 and the second connection port 34 are electrically connected to the processor 31. The first connection port 33 is adapted to be connected to the strip connection port 13 to electrically connect the two. The second connection port 34 can be connected to the key connection port 23 to electrically connect the two. In other possible embodiments, the detection host has an adapter on which at least one of the first and second connection ports is disposed. After the first and second connection ports 33, 34 are electrically connected to the strip connection port 13 and the key connection port 23, respectively, the processor 31 can be used to read the detection parameters of the memory chip and activate the heavy metal detection zone according to the detection parameters, so that the heavy metal detection zone can generate an electrochemical reaction with the possibly existing target heavy metal. The processor 31 can therefore obtain the detection signal and, after the detection, displays the detection result on the host screen 32, so that the operator can obtain the heavy metal concentration data of the sample right at the sampling site. The host screen 32 can be, for example, a touch screen and provides a human-machine interface that can be clicked by the operator. The human-machine interface illustratively has a graphical icon 35 representing the action to startup the detection. When the operator clicks on the graphical icon 35, the processor 31 starts to activate the heavy metal detection zone and performs subsequent detection operations.

In order for the operator to identify what is the target heavy metal of the detection strip 10 and the detection key 20, the processor 31 can read the identification information of the target heavy metal stored in the memory chip 21 and display that on the host screen before performing the detection. For example, in the embodiment, the host screen 32 displays the element symbol “As” of the target heavy metal, indicating that the detection strip 10 is used for detecting arsenic.

In addition, in order to further prevent detection errors, the detection strip 10 may further have an identification unit 14, which is electrically connected to the first connection port and is stored with verification information in a digital manner. The processor 31 further verifies whether the identification information stored in the memory chip 21 matches the verification information or not before activating the heavy metal detection zone 11, and the processor 31 activates the heavy metal detection zone 11 only when the two match. Otherwise the processor 31 lets the host screen 32 to show an error warning without activate the heavy metal detection zone. The identification unit can be, for example, an identification chip.

The electrochemical reaction is induced, for example, by anodic stripping voltammetry, which includes two reaction steps. Taking arsenic as an example, trivalent arsenic in the solution to be tested receives electrons and is reduced to zero-valent arsenic in an acidic environment. The zero-valent arsenic is then deposited on the surface of the heavy metal detection zone, and its reaction formula, hereinafter referred to as the first reaction, is as follows:

As^{III}(OH)₃+3H⁺+3e⁻→As⁰±3H₂O

In the second reaction step, the zero-valent arsenic deposited on the surface of the heavy metal detection zone emits electrons to the surface of the detection strip, and then oxidizes to trivalent arsenic and returns to the solution to be tested. The reaction formula thereof, hereinafter referred to as the second reaction, is as follows:

As⁰+3H₂O→As^{III}(OH)₃+3H⁺+3e⁻

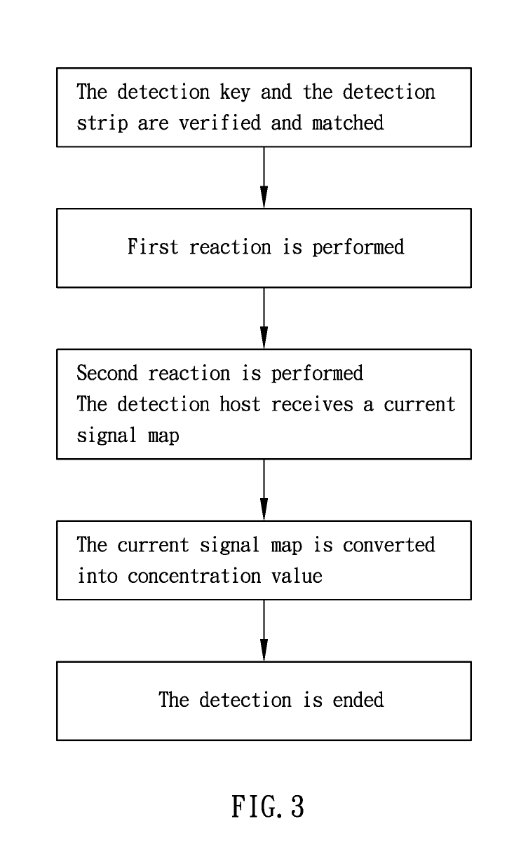

As an exemplary step, the aforementioned detecting operation may include a verification step as shown in FIG. 3, which is initiated after the operator clicks on the graphical icon 35 that indicates the initiation of the detection.

After the processor verifies that the identification information stored in the detection key matches the verification information contained in the detection strip, the processor activates the heavy metal detection zone according to the optimal detection parameters built in the detection key. The possibly existing target heavy metal can sequentially performs the first and second reactions. The processor can receive a current signal map fed back from the heavy metal detection zone when the second reaction is performed, and then convert into a heavy metal concentration value according to the current signal map. The value is then displayed on the host screen and the detection is ended.

In summary, the detection system of the present embodiment can be used for detecting a variety of heavy metals in aqueous solutions, and is convenient for the operator to carry out rapid detection. The aqueous sample is thus no longer needed to be sent to a laboratory for a delayed, time-consuming and expensive detection. The design that the detection strip and the detection key have the same identification color can help the operator quickly distinguish whether the detection strip and the detection key are correctly matched or not. The identification information of the target heavy metal stored in the detection key can be displayed on the host screen, allowing the operator to perform the second identification. The processor can further re-verify whether the identification information matches the verification information contained in the detection strip or not before the detection operation. That is to say, the present embodiment provides three identification levels to ensure that the detection operation can be correctly performed.

What is claimed is:

1. A handheld heavy metal rapid detection system comprising:
a detection strip, having a heavy metal detection zone, a strip identification zone and a strip connection port, the heavy metal detection zone being adapted to be contacted with a solution to be detected and being adapted for electrochemical reaction with a target heavy metal, the strip connection port being electrically connected to the heavy metal detection zone;
a detection key, having a memory chip, a key identification zone and a key connection port, the memory chip being stored with detection parameters of the target heavy metal, the key identification zone having the same color as the strip identification zone, the key connection port being electrically connected to the memory chip; and
a detection host, having a processor, a host screen, a first connection port and a second connection port, the host screen, the first connection port and the second connection port being electrically connected to the processor, the first connection port being adapted to be electrically connected to the strip connection port, the second connection port being adapted to be electrically connected to the key connection port, the processor being adapted to read the detection parameters from the memory chip, to activate the heavy metal detection zone based on the detection parameters, to acquire a detection signal from the heavy metal detection zone, and to show a detection result on the host screen based on the detection signal.

2. The handheld heavy metal rapid detection system of claim 1, wherein the memory chip is further stored with an identification information of the target heavy metal, the processor is further adapted to read the identification information from the memory chip and to show the identification information on the host screen.

3. The handheld heavy metal rapid detection system of claim 2, wherein the detection strip further has an identification unit carrying a verification information, the processor is adapted to verify whether the identification information stored in the memory chip matches the verification information or not before the processor activate the heavy metal detection zone, the processor is adapted to activate the heavy metal detection zone only when the identification information matches the verification information.

4. The handheld heavy metal rapid detection system of claim 3, wherein the processor is adapted to show an error warning on the host screen without activating the heavy metal detection zone when the identification information does not match the verification information.

5. The handheld heavy metal rapid detection system of claim 2, wherein the identification information shown on the host screen comprises a name of the target heavy metal.
